(19) United States
(12) Patent Application Publication
(10) Pub. No.: US 2010/0280185 A1
(43) Pub. Date: Nov. 4, 2010

(54) HIGHLY-BRANCHED MELAMINE POLYMERS

(75) Inventors: Maxim Peretolchin, Mannheim (DE); Eva Rüba, Zurich (CH); Daniel Schönfelder, Mannheim (DE); Bernd Bruchmann, Freinsheim (DE); Günter Scherr, Ludwigshafen (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/602,802

(22) PCT Filed: Jun. 4, 2008
(86) PCT No.: PCT/EP08/56860

§ 371 Date: Jul. 6, 2010

(30) Foreign Application Priority Data
Jun. 5, 2007 (EP) 07109601.0

(51) Int. Cl. C07D 251/32; C08G 73/10; C08F 283/08

(52) U.S. Cl. 525/418; 544/192; 528/367

(57) ABSTRACT

A process for preparing highly branched melamine polymers, wherein melamine is reacted with 1.5 to 4 mol, per mole of melamine, of one or more diamines or polyamines having at least two primary amino groups, it being possible for up to 25 mol % of the diamines or polyamines to have three or more primary amino groups and for up to 50 mol % of the diamines or polyamines to be replaced by amines having only one primary amino group, in the presence of an acidic catalyst.

The invention relates to a process for preparing highly branched melamine polymers and also to the highly branched melamine polymers themselves.

For the preparation of dendrimeric melamine polymers there are a variety of syntheses known. Dendrimers are highly ordered three-dimensional oilgomeric or polymeric compounds which on account of their defined structure are monodisperse. Dendrimers consist of a core and of a number of dendrimers corresponding to the functionality of the core, which in turn consist of in general outwardly branching repeating units of defined construction and defined sequence. In the case of the melamine dendrimers, both the core and the outer branching sites are formed by melamine units joined to one another via spacer groups,

Dendrimers have a compact structure, a high concentration of functional groups, different functionalities on the surface and inside, a high solubility, and a low viscosity in solution and in the melt. These properties allow a broad range of application for the dendrimers, for example, as rheological additives, molecular containers, molecular recognition agents in chromatography, in catalysis, and in electronics.

DE-A 195 28 882 discloses processes for preparing polymelamine dendrimers by reacting cyanuric halides or cyanuric esters, preferably cyanuric chloride or phenyl cyanurate, with a diamine, and subsequently reacting the product again with a cyanuric halide or cyanuric ester, respectively. The reaction can be repeated two or more times in order to construct two or more generations. Alternatively the cyanuric halide or the cyanuric ester can be reacted with a trifunctional aminomelamine which has been prepared by externally reacting cyanuric chloride or cyanuric ester, respectively, with a diamine in excess.

DE-A 198 21 741 relates to stationary phases for chromatography that has been modified with chemically bonded dendrimers, and also to a process for preparing the stationary phases. In that case a substrate having free NH₂ groups, more particularly an NH₂-modified silica gel is reacted with a cyanuric acid derivative, such as trisphenoxytriazine, and then the product is reacted with a diamine, it being possible for the reaction sequence to be repeated in accordance with the number of generations desired. A similar synthesis is described by E. J. Akota et al. in Advanced Materials 2004, 16, No 12, pages 985 to 989. In that ease an amino-functionalized silicon dioxide surface is reacted alternately with cyanuric chloride and aminomethylpiperazine.

W. Zang et ah, J. Am, Chem, Soc. 2001, 123, 8914 to 8922 describe the convergent synthesis of melamine dendrimers starting from cyanuric chloride and para-aminobenzylamine, E. J. Akoefa et al., Journal of Polymer Science: Part A: Polymer Chemistry, Vol. 43, 168 to 177 (2005) describe the convergent synthesis of melamine dendrimers starting from cyanuric chloride and piperazine. W. Zang et al., Macromolecules 2002, 35, 9015 to 9021 describe the convergent synthesis of melamine dendrimers by reacting cyanuric chloride, n-bufylamlne, p-amino-benzylamine, and piperazine.

All of the synthesis pathways described include a labor-intensive multistage organic synthesis. The use of cyanuric chloride leads to high chloride concentrations, thereby necessitating costly and inconvenient cleaning operations after each step of synthesis.

EP-A 1 114 818 describes a process for preparing alkylenebismelamines by reacting melamine with alkylenediamines In the presence of an acidic catalyst, the melamine:alkylenediamine molar ratio being at least 2:1. Acidic catalysts specified include hydrochloric acid, nitric acid, sulfuric acid, phosphoric acid, p-toluenesulfonic acid, and methanesulfonic acid, which can also be used in the form of their ammonium salts. The product in that case essentially comprises dimeric triazine derivatives.

DE-A 38 11 420 describes the preparation of aminoalkyl- or aminoaryl-melamines by reacting diamine with melamine in a molar ration≧10:1 in the presence of an acidic catalyst. The product there comprises essentially monomeric triazine derivatives.

It is an object of the present invention to provide an easy-to-implement process for preparing highly branched melamine polymers.

This object is achieved by means of a process for preparing highly branched melamine polymers wherein melamine is reacted with 1.5 to 4 mol, preferably 1.5 to 3.5 mol, and more preferably 2 to 3 mol, per mole of melamine, of one or more diamines or polyamines having at least two primary amino groups, it being possible for up to 25 mol % of the diamines or polyamines to have three or more primary amino groups and for up to 50 mol % of the diamines or polyamines to be replaced by amines having only one primary amino group, in the presence of an acidic catalyst.

Suitable diamines or polyamines having at least two reactive primary ammo groups are generally linear or branched alkylenediamines, cycloalkylenediamines, aromatic or araliphatic diamines, polyoxyalkylenediamines or polyalkylenepolyamines. These amines may contain heteroatoms in the carbon chain or in the carbon framework (as in the case of polyoxyalkylenediamines or polyalkylenepolyamines) and may also, if appropriate, be substituted, with OH groups, for example.

Examples of suitable diamines having two primary amino groups are ethylenediamine, 1,2- and 1,3-propylenediamine, 2,2-dimethyl, 1,3-propanediamine, butylensdiamine, pentanediamine, hexamethylenediamine, heptanediamine, octanediamine, nonane-diamine, decanediamine, dodecanediamine, hexadecanediamine, tolylenediamine, xylylenediamine, diaminodiphenylmethane, diaminodicyclohexylmethane, phenylene-diamine, cyclohexylenediamine, diaminodiphenyl sulfone, isophoronediamine, bis(aminomethyl)cyclohexane, 2-butyl-2-ethyl-1,5-pentamethylenediamine, 2,2,4- or 2,4,4-trimethyl-1,6-hexamethylenediamine, 2-aminopropylcyclohexylamine, 3(4)-amino-methyl-1-methylcyclohexylamine, 1,4-diamino-4-methylpentane, amine-terminated polyoxyalkylene polyols (known as Jeffamines) or amine-terminated polytetra-methylene glycols. Besides the primary amino groups the diamines may contain secondary or tertiary amino groups. Examples are diethylenetriamine, 3-(2-amino-ethyl)aminopropylamine (N3-amine), dipropylenetriamine, N,N-bis-(3-aminopropyl)-methylamine and (N,N-bis(3-aminopropyl)ethylenediamine (N4-amine) and tetra-ethylenepentamine.

Preferred diamines having two primary amino groups are butylenediamine, pentanediamine, hexamethylenediamine, toluylenediamine, xylylenediamine, diamino-diphenylmethane, diaminodicyclohexylmethane, phenylenediamine, cyclohexylene-diamine, diaminediphenyl sulfone, isophoronediamine, bis(aminomethyl)cyclohexane, amine-terminated polyoxyalkylene polyols (Jeffamines, examples being 4,9-dioxa-dodecane-1,12-diamine or 4,7,10-trioxatridecane-1,13-diamine), and amine-terminated polytetramethylene glycols.

Particularly preferred diamines or polyamines having two primary amino groups are hexamethylenediamine, diaminodicyclohexylmethane, isophoronediamine, ethylene-diamine, 1,2- and 1,3-propylenediamine, 2,2-dimethyl-1,3-propanediamine, amine-terminated polyoxyalkylene polyols and amine-terminated polytetramethylene glycols.

In order to set a high degree of branching of the highly branched melamine polymers it is possible for up to 25 mol % of the 1 to 4 mol of amines reacted per mole of melamine to be polyamines having three or more primary amino groups. Examples are tris(aminoethyl)amine, tris(aminopropyl)amine, tris(aminohexyl)amine, trisamino-hexane, 4-aminomethyl-1,8-octamethylenediamine, trisaminononane or amine-terminated polyoxyalkylene polyols with a functionality of 3 or more (Jeffamines, examples being polyetheramine T403 or polyetheramine T5000).

Up to 50 mol % of the 2 to 3 mol of amines used per mole of melamine may be one or more amines having only one (reactive) primary amino group. Examples are n-pentylamine, tert-butylamine, n-hexylamine, 3-methoxypropylamine, 2-ethoxy-ethylamine, 3-ethoxypropylamine, 3-(2-ethylhexyloxy)propylamine, monoethanolamine, 3amino-1-propanol, isopropanolamine, 5-amino-1 -pentanol, 2-(2-aminoethoxy)ethanol, aminoethylethanolamine and N-(2-hydroxyethyl)-1,3-propanediamine.

Suitable acidic catalysts Include all strong and moderately strong protic acids, examples being hydrofluoric acid, hydrochloric acid, hydrobromic acid, nitric acid, sulfuric acid, phosphoric acid, amidosulfonic acid, thiocyanic acid, p-toluenesulfonic acid, or methanesulfonic acid. The acids can be added either in free form or as melamine or amine salt (diamine salt or polyamine salt). In one preferred embodiment the acid is added as ammonium salt, i.e., in the form of ammonium chloride, ammonium sulfate, ammonium bromide, etc., for example. Generally speaking use is made, per mole of melamine, of 0.001 to 3 mol, preferably 0.05 to 1 mol of catalyst.

In lieu of the specified protic acids, the reaction is also catalyzed by Lewis acids, such as boron trifluoride, aluminum chloride, zinc(IV) chloride, antimony(V) fluoride or iron(III) bromide.

The process of the invention is advantageously carried out by heating a mixture of melamine, the amine or amines, acidic catalyst, and, if appropriate, a solvent at temperatures from 120 to 250° C. Amines whose boiling point is high enough to allow a sufficiently rapid reaction are generally reacted under atmospheric pressure. Where the boiling point of the amine Is below 180° C., however, it is generally advantageous to carry out the reaction at a higher temperature under superatmospheric pressure. Even with amines having boiling points above 180° C. it may under certain circumstances be advantageous to carry out the reaction under superatmospheric pressure, in order to achieve a higher reaction temperature and hence a higher reaction rate. The ammonia formed is removed from the reaction mixture, by means for example of distillation under a pressure of 1 to 20 bar.

The reaction can be carried out if appropriate in a solvent. Examples of suitable solvents are polyols, such as ethylene glycol, 1,2-propylene glycol, diethylene glycol, triethylene glycol or polyethylene glycol.

In one preferred embodiment the operation takes place in the absence of an additional solvent. In one particularly preferred embodiment the amine or amine mixture is introduced as an initial charge and heated, and melamine is added, in one portion or distributed over a number of portions. The mixture is left to react until the desired viscosity has been reached.

When a viscosity of in general 500 to 100,000 mPas has been reached, measured at 100° C., the reaction is discontinued by lowering the temperature to a level in the range of in general 20 to 50° C.

To remove the acidic catalyst the product can be subsequently treated with a strong organic base such as aqueous sodium hydroxide solution or aqueous potassium hydroxide solution. In those cases, sodium or potassium salts precipitated are generally removed by filtration. Small amounts of catalyst may remain in the product.

In general the products according to the invention have a number-average molecular weight Mₙ in the range from 1500 to 20,000 g/mol preferably 2000 to 10,000 g/mol, and a weight-average molecular weight in the range M_{w} in the range from 1500 to 150,000 g/mol, preferably 2000 to 50,000 g/mol. Number-average and weight-average molecular weight are determined by GPC in hexafluoroisopropanol (HFIP) in accordance with the instructions described in the examples.

The highly branched melamine polymers of the invention that are obtained can be used as a catalyst for polyurethane synthesis, curing agent for epoxy resins, starter substances for alkoxylations with ethylene oxide or propylene oxide for preparing polyols, or DNA transection agents.

The present invention also provides for the use of the highly branched melamine polymers as curing agents for spoxy resins.

### Epoxy Resins

With regard to the epoxy resins for curing there is no restriction whatsoever on the inventive use.

The majority of commercial uncured epoxy resins are prepared by coupling epichlorohydrin onto compounds which possess at least two reactive hydrogen atoms, such as polyphenols, monoamines and diamines, aminophenols, heterocyclic imides and amides, aliphatic diols or polyols or dimeric fatty acids. Epoxy resins derived from epichlorohydrin are referred to as glycidyl-based resins.

The majority of epoxy resins available commercially at the present time derive from the diglycidyl ether of bisphenol A (DGEBA resins) and possess the general formula

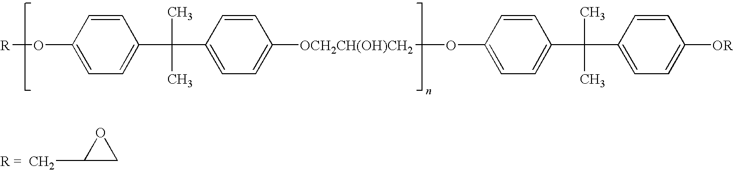

In which n stands for 0 to approximately 40.

Other important epoxy resins are phenol-based and cresol-based epoxy novolaks, examples being epoxy resins which derive from the diglycidyl ether of bisphenol F. Novolaks are prepared by the acid-catalyzed condensation of formaldehyde and phenol or cresol. The epoxidation of the novolaks leads to epoxy novolaks.

Other classes of glycidyl-based epoxy resins derive from glycidyl ethers of aliphatic diols, such as butane-1,4-diol, hexane-1,6-diol, pentaerythritol or hydrogenated bisphenol A; aromatic glycidylamines, an example being the triglycidyl adduct of p-aminophenol or the tetraglycidylamine of methylenedianilide; heterocyclic glycidylimides and amides, e.g., triglycidyl isocyanurate; and glycidyl esters, such as the diglycidyl ester of dimeric linoleic acid, for example.

The epoxy resins may also derive from other epoxides (non-glycidyl ether epoxy resins). Examples are the diepoxides of cycloaliphatic dienes, such as 3,4-epoxycyclohexylmethyl 3,4-epoxycyclohexanecarboxylate and 4-epoxyethyl-1,2-epoxycyclohexane.

The condensation products used in accordance with the invention are particularly suitable for the curing of epoxy resins based on glycidyl polyethers of bisphenol A. bisphenol F, and novolak resins.

Curing agents used in accordance with the Invention are the highly branched melamine polymers. They can be used as sole curing agents; it is, however, also possible to use them in combination with one or more conventional curing agents for epoxy resins.

The conventional curing agents include aliphatic and aromatic polyamines, polyamidoamines, urons, amides, guanidines, amino resins and phenolic resins, polycarboxylic polyesters, dihydroxy and polyhydroxy compounds, thiols, imidazoles, imidazolines, and certain isocyanates, and also latent polyfunctional curing agents.

Polyamine curing agents crosslink epoxy resins through reaction of primary or secondary amino functions of polyamines with terminal epoxide groups of the epoxy resins. Suitable polyamines are, for example, aliphatic polyamines such as ethylene-diamine, 1,2- and 1,3-propylenediamine, neopentanediamine, hexamethylenediamine, octamethylenediamine, 1,10-diaminodecane, 1,12-diaminododecane, diethylene-triamine, triethylenetetramine, tetraethylenepentamine, and the like; cycloaliphatic diamines, such as 1,2-diaminocyclohexane, 1,3-bis(aminomethyl)cyclohexane, 1-methyl-2,4-diaminocyclohexane, 4-(2-aminopropan-2-yl)-1-methylcyclohexane-1-amine, isophoronediamine, 4,4′-diaminodicyclohexylmethane, 3,3′-dimethyl-4,4′-diaminodicyclohexylmethane, norbornanediamine, menthanediamine, menthene-diamine and the like; aromatic diamines, such as tolylenediamine, xylylenediamine, more particularly meta-xylylenediamine, bis(4-aminophenyl)methane (MDA or methylenedianiline), bis(4-aminophenyl) sulfone (also known as DADS, DDS or dapsone), and the like; cyclic polyamines, such as piperazine, N-aminoethylpiperazine and the like; polyetherdiamines, example being the reaction product of polypropylene oxide or polyethylene oxide or butylene oxide or pentylene oxide or poly(1,4-butanediol) or polytetrahydrofuran or mixtures of the 5 last-mentioned alkylen oxides with propylene oxide with ammonia, e.g., 4,7,10-trioxatridecane-1,3-diamine, 4,7,10-trioxatridecane-1,13-diamine, XTJ-500, XTJ-501, XTJ-511, XTJ-542, XTJ-559, XTJ-566, XTJ-568 (Huntsman), 1,8-diamino-3,6-dioxaoctane (XTJ-504 from Huntsman), 1,10-diamino-4,7-dioxadecane (XTJ-590 from Huntsman), 1,12-diamino-4,9-dioxadodecane (BASF), 1,3-diamino-4,7,10-trioxatridecane (BASF), polyetheramine T 5000, Jeffamines and the like; and polyamide diamines (amidopolyamines), which are obtainable through the reaction of dimeric fatty acids (e.g., dimeric linoleic acid) with low molecular mass polyamines, such as diethylenetriamine or triethylenetetramine.

A further class of suitable curing agents are those known as urons (urea derivatives), such as 3-(4-chlorophenyl-1,1-dimethylurea (monuron), 3-(3,4-dichlorophenyl)-1,1-dimethylurea (diuron), 3-phenyl-1,1-dimethylurea (fenuron), 3-(3-chloro-4-methylphenyl)-1,1-dimethylurea (chlorotoluron), and the like,

Suitable curing agents are also carbamides, such as tolyl-2,4-bis(N,N-dimethylcarbamide), and tetraalkylguanidines, such as N,N,N′,N′-tetramethylguanidine.

Melamine-, urea-, and phenol-formaldehyde adducts, which are also referred to as amino resins or phenolic resins, respectively, form a further class of epoxide curing agents.

Polycarboxylic polyesters as curing agents are being employed increasingly in powder coatings. The crosslinking takes place by virtue of the reaction of the free carboxyl groups with the epoxide groups of the epoxy resin.

Further polyfunctional curing agents comprise aromatic compounds having two or more hydroxy; groups, Examples of such are resins obtainable by the reaction of phenol or alkylated phenols, such as cresol, with formaldehyde, examples being phenol novolaks, cresol novolaks and dicyclopentadiene novolaks; furthermore, resins of nitrogen-containing heteroaromatics, such as benzoguanamine-phenol-formaldehyde resins or benzoguanamine-cresol-formaldehyde resins, acetoguanamine-phenol-formaldehyde resins or acetoguanamine-cresol-formaldehyde resins, and melamine-phenol-formaldehyde resins or melamine-cresol-formaldehyde resins, and also hydroxylated arenes, such as hydroquinone, resorcinol, 1,3,5-trihydroxybenzene, 1,2,3-trihydroxybenzene (pyrogallol), 1,2,4-trihydroxybenzene (hydroxyhydroquinone), 3,4,5-trihydroxybenzoic acid (gallic acid) or derivatives thereof, 1,8,9-trihydroxyanthracene, (dithranol or 1,8,9-anthracenetriol), 1,2,10-trihydroxyanthracene (anthrarobin) and 2,4,5-trihydroxypyrimidine; additionally, alkanes substituted by hydroxylated arenas, such as triphenolmethane, triphenolethane and tetraphenolethane. Further examples are phosphinates and phosphorates derived from hydroquinone and naphthoquinone, as described in WO 2006/034445, hereby fully incorporated by reference.

Further polyfunctional curing agents comprise thiols, imidazoles, such as imidazole, 1-methylimidazole, 2-methylimidazole, 2-ethyl-4-methylimidazole, 1-cyanoethyl-imidazole and 2-phenylimidazole, and imidazolines, such as 2-phenylimidazoline.

Blocked isocyanates have more recently been used as latent curing agents for water-based coatings,

Dicyandiamide (dicy), HN═C(NH₂)(NHCN), is a latent polyfunctional curing agent frequently employed in powder coatings and electrical laminates.

Also suitable are reaction products of dicy with amines, known as bisguanidines, such as HAT 2844 from Vantico.

Further suitable latent polyfunctional curing agents are boron trifluoride-amine adducts such as BF₃-monoethylamine, and quaternary phosphonium compounds.

Preferred conventional curing agents are selected from the abovementioned aliphatic polyamines, cycloaliphatic diamines, polyetheramines, and mixtures thereof.

If one or more conventional curing agents are used alongside the curing agents (i) to (vi) employed in accordance with the invention, the weight ratio of the total amount of all the curing agents (i) to (vi) used to the total amount of all the conventional curing agents used is preferably from 1:1000 to 100:1, more preferably from 1:100 to 50:1, and more particularly 1:50 to 30:1.

The curing agents (i.e., the entirety of all curing agents used in accordance with the invention and any conventional curing agents used) are employed in amounts such that the ratio of the number of all the reactive groups (in the case of the curing agents used in accordance with the invention, these are all the hydrogen atoms on primary and secondary amino functions) to the number of all the epoxide groups in the epoxy resin is 2:1 to 1:2, preferably 1.5:1 to 1:1.5, and more particularly about 1:1. At a stoichiometric ratio of approximately 1:1, a cured resin having optimum thermoset properties is obtained. Depending on the desired properties of the resin after crosslinking, however, it may also be sensible to use curing agent and epoxy resin in different proportions of the reactive groups.

The number of epoxide groups in the epoxy resin is cited as what is called the epoxide equivalent. The epoxide equivalent is determined in accordance with DIN 16 945.

The number of reactive groups in the curing agent is calculated, in the case of amine curing agents, which encompasses the condensation products used in accordance with the invention, via the amine number in accordance with DIN 18945.

The curing of the epoxy resins is accomplished, preferably, thermally by heating of the mixture of epoxy resin and curing agent to a temperature of preferably 5 to 150° C., more preferably 20 to 150° C., even more preferably from: 25 to 125° C., and more particularly 30 to 100° C., in the lower temperature range (5 to about 25° C.), which indeed corresponds to the ambient temperature that is normally prevailing, it is, of course, sufficient to mix epoxy resin and curing agent. Which temperature is suitable depends on the particular curing agents and epoxy resins and on the desired cure rate, and can be determined in each individual case by the skilled worker on the basis, for example, of simple preliminary tests.

Alternatively the curing takes place with, preferably, microwave induction.

The invention is illustrated in more detail by the examples below.

### EXAMPLES

### Example 1

2.8 mol of hexamethylenediamine are charged to a reaction vessel and melted. Under a nitrogen atmosphere, 0.1 mol of ammonium chloride is added as catalyst. The mixture is heated slowly with stirring to 60° C., In the course of which gas evolution is observed. In order to assist the removal of ammonia gas that forms, the system is flushed with nitrogen. Then 0.2 mol of melamine Is added and the mixture is heated at 200° C. until all of the melamine has dissolved. Then a further 0.2 mol of melamine are added and the reaction is continued at 200°C. This operation is repeated a further 3× until the total amount of melamine added is 1 mol. The reaction is continued at 200° C. until the oil formed has reached a viscosity of 35,000 mPas, measured at 50° C. The reaction mixture is subsequently cooled to room temperature. The highly branched melamine polymer is obtained as a viscous yellow oil.

Analytical characterization of the product gives the following values:

amine number: 517 mg KOH/g;
primary amine: 8.8 g KOH/g;
secondary amine: 0.8 g KOH/g;
tertiary amine: 3.2 g KOH/g:
chloride concentration: 0.86 g/100 g;
number-average molecular weight: Mₙ: 2300 g/mol;
weight-average molecular weight: M_{w}: 6800 g/mol;
polydispersity M_{w}/Mₙ: 3.0 (Mₙ, M_{w} determined by GPC in HFIP against MMA standard, see below);

viscosity: 36.100 mPas at 50° C.;

soluble in hexafluoroisopropanol, DMSO, water (pH=5 to 8), ethylene glycol, DMF.

The molecular weight determination took place in hexafluoroisopropanol with addition of 0.05% by weight of potassium trifluoroacetate as the mobile phase in a “PL HFIP gel linear” column from Polymer Laboratories with a flow rate of 0.5 ml/min. Calibration took place with PMMA standards having molecular weights of 800 to 1,820,000 g/mol.

### Example 2

1.8 mol of hexamethylenediamine are charged to a reaction vessel and melted. Under a nitrogen atmosphere, 1 mol of diaminodicyclohexylmethane and 0.33 mol of ammonium chloride are added. The mixture is heated slowly with stirring to 60° C. Then 0.5 mol of melamine are added, in the course of which evolution of gas is observed. To remove the ammonia gas released, the system is flushed with nitrogen. It is heated further at 200° C. When all of the melamine has dissolved, a further 0.5 mol of melamine is added and the reaction is continued at 200° C. Following complete dissolution of the melamine, the reaction is continued until the reaction product has reached a viscosity of 16,000 mPas, measured at 100° C. The resulting dendrite melamine polymer is a yellow amorphous solid at room temperature.

Analysis of the product gave the following values:

amine number: 375 mg KOH/g;
primary amine: 49 g/100 g
secondary amine: 2.2 g/100 g;
tertiary amine: 2,2 g/100 g;
Mₙ=1790 g/mol;
M_{w}=9300 g/mol;

### M_{w}/Mₙ=5.2;

viscosity: 16,300 mPas at 100° C.;
soluble in hexafluoroisopropanol, DMSO, water (pH=5 to 8), DMF.

### Example 3

2.8 mol of isophoronediamine are charged to a reaction vessel. Under a nitrogen atmosphere, 0.05 mol of ammonium chloride and 0.2 mol of melamine are added. The mixture is heated slowly with stirring to 60° C. In the course of heating, evolution of gas is observed. To remove the ammonia formed, the system is flushed with nitrogen. Then the mixture is heated at 200° C. until all of the melamine has dissolved. Subsequently a further 0.2 mol of melamine is added and the reaction is continued at 200° C. The procedure is repeated a further 3× until the total amount of melamine added is 1 mol. When all of the melamine has dissolved, the reaction is continued until the total reaction time amounts to 40.5 h. The resulting dendrite melamine polymer is a vitreous amorphous solid.

Analysis of the product gave the following values:

amino number: 368 mg KOH/g;
primary amine: 2.8 g KOH/g
secondary amine: 4.0 g KOH/g;
tertiary amine: 2.5 g KOH/g;
Mₙ=5000 g/mol;
M_{w}=9500 g/mol;

### M_{w}/Mₙ=1.9;

soluble in hexafluoroisopropanol, DMSO, DMF.

### Example 4

4.2 mol of N,N-bis(3-aminopropyl)ethylenediamine, 0.7 mol of ammonium chloride and 0.3 mol of melamine are charged to a reaction vessel. The mixture is slowly heated to 200° C. In the course of heating, evolution of gas is observed. The reaction mixture is flushed with nitrogen in order to remove ammonia released. When all of the melamine has dissolved, a further 0.3 mol of melamine is added and the reaction is continued at 200° C. The procedure Is repeated a further 3× until the total amount of melamine added is 1.5 mol. When all of the melamine is dissolved, the reaction Is continued to 200° C. until the viscosity of the oil formed is 51,100 mPas, measured at 23° C. The resulting dendrite melamine polymer is a viscous brown oil. This oil is admixed slowly with 0.87 mol of 50% strength aqueous sodium hydroxide solution, and the mixture is heated to 75° C. The solid formed is isolated by filtration.

Analysis of the product obtained gave the following values:

amino number: 368 mg KOH/g;
primary amine: 4.0 g KOH/g
secondary amine: 3.4 g KOH/g;
tertiary amine: 6.0 g KOH/g;
M_{w}=6000 g/mol;
Mₙ=2700 g/mol;

### M_{w}/Mₙ=2.2;

viscosity before addition of NaOH: 51,100 mPas at 23° C.;
soluble in hexafluoroisopropanol, DMSO, water, ethylene glycol, DMF.

### Example 5

Example 6 is carried out in accordance with example 1 with the difference that the melamine:hexamethylenediamine:ammonium chloride molar ratio is 1:2.5:0.1. The reaction is discontinued on reaching a viscosity of 50,800 mPas, measured at 75° C.

Analysis of the product gives the following values:

amine number: 465 mg KOH/g;

primary amine: 5.5 g KOH/g

secondary amine; 2.5 g KOH/g;

tertiary amine: 3.6 g KOH/g;

Mₙ=2400 g/mol;
M_{w}=8800 g/mol;

### M_{w}/Mₙ=3.7;

soluble in hexafluoroisopropanol, DMSO, water (pH=5 to 8), DMF, ethylene glycol, ethanol.

### Example 6

1.1 mol of polyetheramine D400 from BASF AG, DE, (polyetherolamine with terminal NH₂ groups; CAS No. 9046-10-0) and 0.25 mol of ammonium chloride are charged to a reaction vessel and this initial charge is heated slowly with stirring to 60° C. During heating, evolution of gas is observed. Ammonia gas released is removed by flushing with nitrogen. 0.17 mol of melamine is added and the mixture is heated to 200° C. When all of the melamine is dissolved, a further 0.17 mol of melamine is added, and the reaction is continued at 20° C. Finally, once again, 0.16 mol of melamine is added. When all of the melamine is dissolved, the reaction Is continued at 200° C. until the oil formed has reached a viscosity of 4500 mPas, measured at 100° C. The dendrite melamine polymer obtained is a viscous yellow oil.

Analysis of the product gives the following values:

total amine content: 1.7 g/100 g;
primary amine: 0.4 g/100 g;
secondary amine: 0.1 g/100 g:
tertiary amine: 1.1 g/100 g;
Mₙ=17800 g/mol;
M_{w}=38500 g/mol;

### M_{w}/Mₙ=2.2;

soluble in hexafluoroisopropanol, DMSO, water (pH=5 to 8), DMF.

### Example 7

778.7 g (4.5 mol) of N,N-bis(3-aminopropyl)ethylenediamine, 40.1 g of ammonium chloride, and 50.4 g (0.4 mol) of melamine are mixed and the mixture is heated slowly under nitrogen to 200° C. In the course of the heating procedure, gas is evolved (ammonia). Following complete dissolution of the melamine, the mixture is cooled to 100° C., 50.4 g (0.4 mol) of melamine are added, and the mixture is again heated to 200° C. This procedure is repeated until a total of 189 g (1.5 mol) of melamine have been added. The reaction mixture is held at 200° C. under nitrogen until the viscosity (23° C.) has reached about 50,000 mPa·s. The viscous brown oil obtained is admixed slowly at 75° C. with 68.4 g of NaOH (in the form of a 50% strength aqueous solution) and the precipitate formed is removed by filtration. This gives an oil having the following properties:

Mₙ: 2700; M_{w}: 6000; PD: 2.2; amine number: 539 mg KOH/g; viscosity (23°): 6430 mPa·s

### Example 8

927.2 g (5.4 mol) of isophoronediamine, 5.22 g of ammonium chloride, and 49.1 g (0.4 mol) of melamine are mixed and the mixture is heated slowly to 200° C. under nitrogen, in the course of the heating procedure, gas is evolved (ammonia). Following complete dissolution of the melamine the mixture is cooled to 100° C., 49.1 g (0.4 mol) of melamine are added, and the mixture is again heated to 200° C. This procedure is repeated until a total of 245.5 g (1.9 mol) of melamine have been added. The reaction mixture is held at 200° C. under nitrogen for 40.5 h. Cooling to room temperature gives a beige, glasslike solid having the following properties:

Mₙ: 5000; M_{w}: 9500; PD: 1.9; amine number: 368 mg KOH/g

### Example 9

325.6 g (2.8 mol) of hexamethylenediamine, 5.35 g of ammonium chloride, and 25.2 g (0.2 mol) of melamine are mixed and the mixture is heated slowly to 200° C. under nitrogen. In the course of the heating procedure, gas is evolved (ammonia). Following complete dissolution of the melamine the mixture is cooled to 100° C. 25.2 g (0.2 mol) of melamine are added, and the mixture is again heated to 200° C. This procedure is repeated until a total of 126 g (1.0 mol) of melamine have been added. The reaction mixture is held at 200° C. under nitrogen until the viscosity (50° C.) has reached about 35,000 mPa·s. Cooling to room temperature gives an oil having the following properties:

Mₙ: 2300; M_{w}: 6800; PD: 3.0; amine number: 517 mg KOH/g; viscosity (50° C.): 36,100 mPa·s

### Curing of Epoxy Resins

The products from the preparation examples were used either as they are or in a mixture with a mix of 70% by weight Jeffamine D-230 (polyetheramine from Huntsman Corp.; difunctional, amine-terminated polyetherol; Mₙ approximately 230 g/mol) and 30% by weight of isophoronediamine, referred to below as D-230/IPDA.

The epoxy resin used was Epilox® A 19-00 (Leuna-Hanre GmbH; Leuna, Germany) (epoxide equivalent according to DIN 16,945:182-192 g/equiv.; viscosity (25° C.) according to DIN 53 015 9009-13,000 mPa·s; density (20° C.) according to DIN 53,217 T.4 1.17 g/cm³; Gardner color number; DIN ISO 4630<2).

### General Procedure

1 g of the product from one of the above preparation examples, or a mixture thereof with D-230/IPDA in the weight ratio indicated below, was admixed with Epilox® A-1900. The amount of Epilox was chosen so that there was one reactive hydrogen atom per epoxide equivalent. The number of reactive protons was calculated from the amine number in accordance with DIN 16945.

The mixture was poured into different molds, degassed at room temperature in an ultrasound bath, and cured In a drying oven at 40° C. for 16 h.

All of the cured products were hard and clear or slightly opaque.

### Example 10

Curing agent: product from example 7
Amount of curing agent: 5 g

### Amount of Epilox®: 17.50 g

Cured product pale yellow, clear

### Example 11

Curing agent: mixture: 10% by weight product from example 7 and 90% by weight D-230/IPDA. The mixture had an amine number of 513 mg KOH/g
Amount of curing agent: 5 g

### Amount of Epilox®: 16.65 g

Cured product: colorless, cloudy

### Example 12

Curing agent: mixture; 10% by weight product from example 8 and 90% by weight D-230/IPDA. The mixture had an amine number of 495 mg KOH/g
Amount of curing agent: 5 g

### Amount of Epilox®: 16.05 g

Cured product: colorless, cloudy

Example 13

Curing agent: mixture; 10% by weight product from example 8 and 90% by weight D-230/IPDA. The mixture had an amine number of 505 mg KOH/g
Amount of curing agent; 5 g

### Amount of Epilox®: 16.40 g

Cured product: colorless, slightly cloudy

1. A process for preparing highly branched melamine polymers, wherein melamine is reacted with 2 to 3 mol, per mole of melamine, of one or more diamines or polyamines having at least two primary amino groups, it being possible for up to 25 mol % of the diamines or polyamines to have three or more primary amino groups and for up to 50 mol % of the diamines or polyamines to be replaced by amines having only one primary amino group, in the presence of an acidic catalyst.

2. The process according to claim 1, wherein the reaction is discontinued by temperature reduction when a viscosity of in general 500 to 100,000 mPas is reached, measured at 100° C.

3. Highly branched melamine polymers obtainable by the process according to claim 1.

4. The highly branched melamine polymers according to claim 3, having a number average molecular weight Mₙ in the range from 1500 to 20,000 g/mol and a weight-average molecular weight M_{w} in the range from 1500 to 150,000 g/mol.

5. The use of highly branched melamine polymers according to claim 3 as curing agent for epoxy resins.

6. Highly branched melamine polymers obtainable by the process according to claim 2.

7. The use of highly branched melamine polymers according to claim 4 as curing agent for epoxy resins.
